(19) United States
(12) Patent Application Publication
(10) Pub. No.: US 2014/0180158 A1
(43) Pub. Date: Jun. 26, 2014

(54) SENSING DEVICE FOR MEASURING ELECTROENCEPHALOGRAM

(71) Applicants: LITE-ON ELECTRONICS (GUANGZHOU) LIMITED, (US); LITE-ON TECHNOLOGY CORPORATION, TAIPEI CITY (TW)

(72) Inventors: CHI-HSIANG CHENG, NEW TAIPEI CITY (TW); LIANG-YI LIU, TAIPEI CITY (TW)

(73) Assignees: LITE-ON TECHNOLOGY CORPORATION, TAIPEI CITY (TW); LITE-ON ELECTRONICS (GUANGZHOU) LIMITED, GUANGZHOU (CN)

(21) Appl. No.: 13/845,185

(22) Filed: Mar. 18, 2013

(30) Foreign Application Priority Data
Dec. 26, 2012 (CN) 201210574266.8

(51) Int. Cl. A61B 5/0476; A61B 5/00

(52) U.S. Cl. CPC A61B 5/0476; A61B 5/6803
USPC 600/544

(57) ABSTRACT

An exemplary embodiment of the present disclosure provides a sensing device for measuring electroencephalogram (EEG). The sensing device includes an arm, a sensing portion, a multi-directional joint, a connecting base and a headband component. The arm has a front end and a back end opposite to the front end. The back end is coupled to the connecting base. The multi-directional joint is disposed on the front end, and coupled to the sensing portion, so that the sensing portion can rotate relative to the front end of the arm. The headband component includes a plurality of elastic arcuate bands and an anti-slip pad. Each of the elastic arcuate bands has a first side and a second side opposite to the first side. The first side is coupled to the anti-slip pad, and the second side is coupled to the connecting base.

### BACKGROUND

1. Technical Field

The present disclosure relates to a sensing device for measuring electroencephalogram, in particular a headband-type sensing device for measuring electroencephalogram.

2. Description of Related Art

Recently, the common sensing device for measuring electroencephalogram is headband-type design. When using the sensing device, a user wears a headband of the sensing device and adjusts an electroencephalogram sensor against a forehead to get electroencephalogram signals.

FIG. 1 illustrates a front view diagram of a conventional sensing device for measuring electroencephalogram. Please refer to FIG. 1. The conventional sensing device for measuring electroencephalogram includes a headband T, a first electroencephalogram sensor S, a second electroencephalogram sensor V, and a foam anti-slip pad U. The headband T is an arc-shaped plastic solid band, and the end of the headband T is coupled to the foam anti-slip pad U. By using the headband T, the user could wear the conventional sensing device for measuring electroencephalogram. Besides, the most sensing devices use plastic solid headbands, and the plastic solid headband has poor elasticity.

### SUMMARY

An exemplary embodiment of the present disclosure illustrates a sensing device for measuring electroencephalogram, in particular a headband-type sensing device for measuring electroencephalogram.

An exemplary embodiment of the present disclosure illustrates a sensing device for measuring electroencephalogram. The sensing device includes an arm, a sensing portion, a multi-directional joint, a connecting base, and a headband component. The arm has a front end and a back end opposite to the front end, and the back end of the arm is coupled to the connecting base. The multi-directional joint is disposed on the front end of the arm. The sensing portion is coupled to the multi-directional joint, so that the sensing portion can rotate with respect to the front end of the arm. The sensing portion measures electroencephalogram and generates a first electroencephalogram signal. The headband component includes a plurality of elastic arcuate bands and an anti-slip pad. Each of the elastic arcuate bands has a first side and a second side opposite to the first side. The first side is coupled to the anti-slip pad, and the second side is coupled to the connecting base.

To sum up, the present disclosure provides the sensing device, in which the sensing portion is disposed on the arm through the multi-directional joint, and the headband component is coupled to the arm. The elastic arcuate bands and anti-slip pad (which could be T-shaped anti-slip pad) could fit the type of head, so that the sensing device for measuring electroencephalogram could be wear on the head. Therefore, compared with conventional sensing device for measuring electroencephalogram, the present disclosure is not easy to be slack.

In order to further understand the techniques, means and effects of the present disclosure, the following detailed descriptions and appended drawings are hereby referred, such that, through which, the purposes, features and aspects of the present disclosure can be thoroughly and concretely appreciated; however, the appended drawings are merely provided for reference and illustration, without any intention to be used for limiting the present disclosure.

### BRIEF DESCRIPTION OF THE DRAWINGS

The accompanying drawings are included to provide a further understanding of the present disclosure, and are incorporated in and constitute a part of this specification. The drawings illustrate exemplary embodiments of the present disclosure and, together with the description, serve to explain the principles of the present disclosure.

FIG. 1 depicts a front view diagram of a conventional sensing device for measuring electroencephalogram.

FIG. 2A depicts a perspective diagram of a sensing device for measuring electroencephalogram in accordance with the first exemplary embodiment of the present disclosure.

FIG. 2B depicts a front view diagram of a sensing device for measuring electroencephalogram shown in FIG. 2A.

FIG. 2C depicts a vertical view diagram of using a sensing device for measuring electroencephalogram shown in FIG. 2A.

FIG. 3A depicts a perspective diagram of a sensing device for measuring electroencephalogram in accordance with the second exemplary embodiment of the present disclosure.

FIG. 3B depicts a front view diagram of a sensing device for measuring electroencephalogram shown in FIG. 3A.

FIG. 3C depicts a vertical view diagram of using a sensing device for measuring electroencephalogram shown in FIG. 3A.

### DESCRIPTION OF THE EXEMPLARY EMBODIMENTS

Reference will now be made in detail to the exemplary embodiments of the present disclosure, examples of which are illustrated in the accompanying drawings. Wherever possible, the same reference numbers are used in the drawings and the description to refer to the same or like parts.

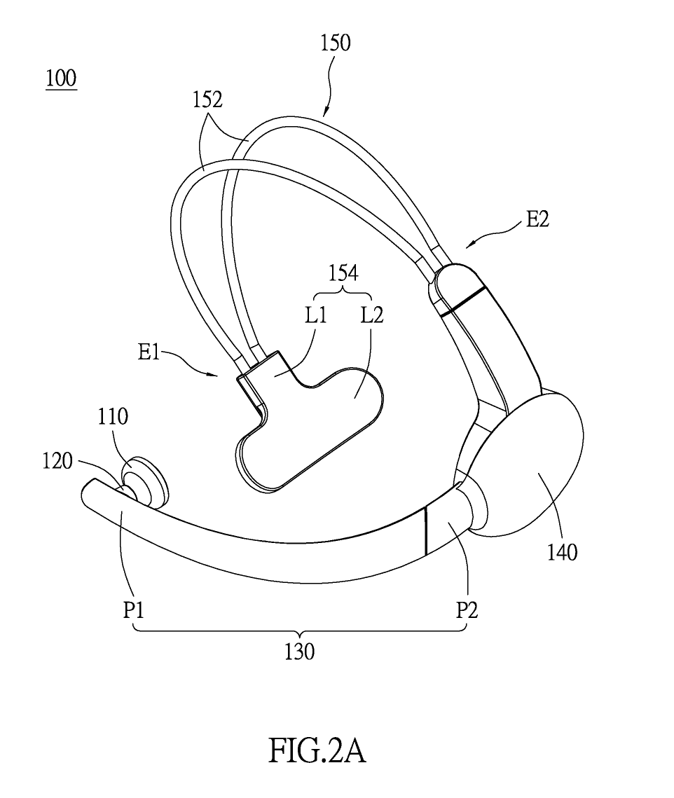

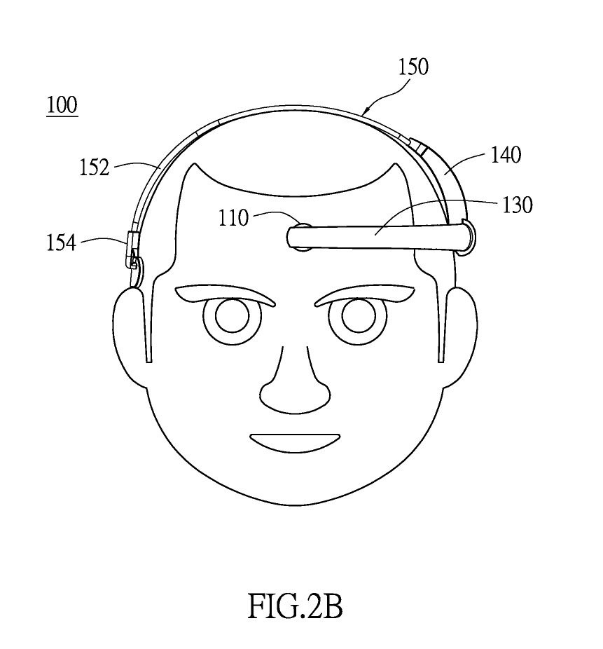

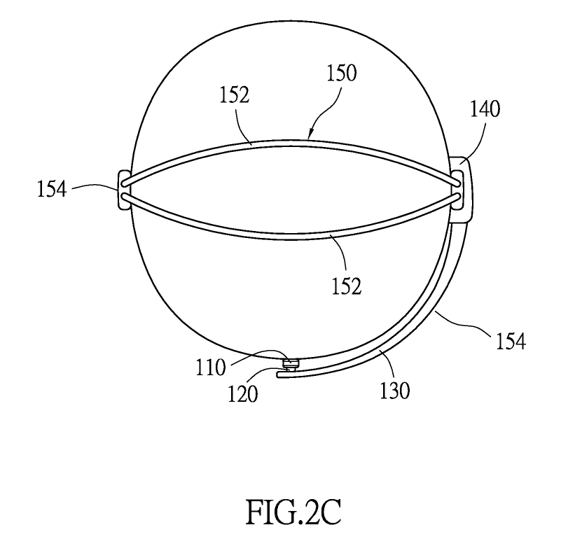

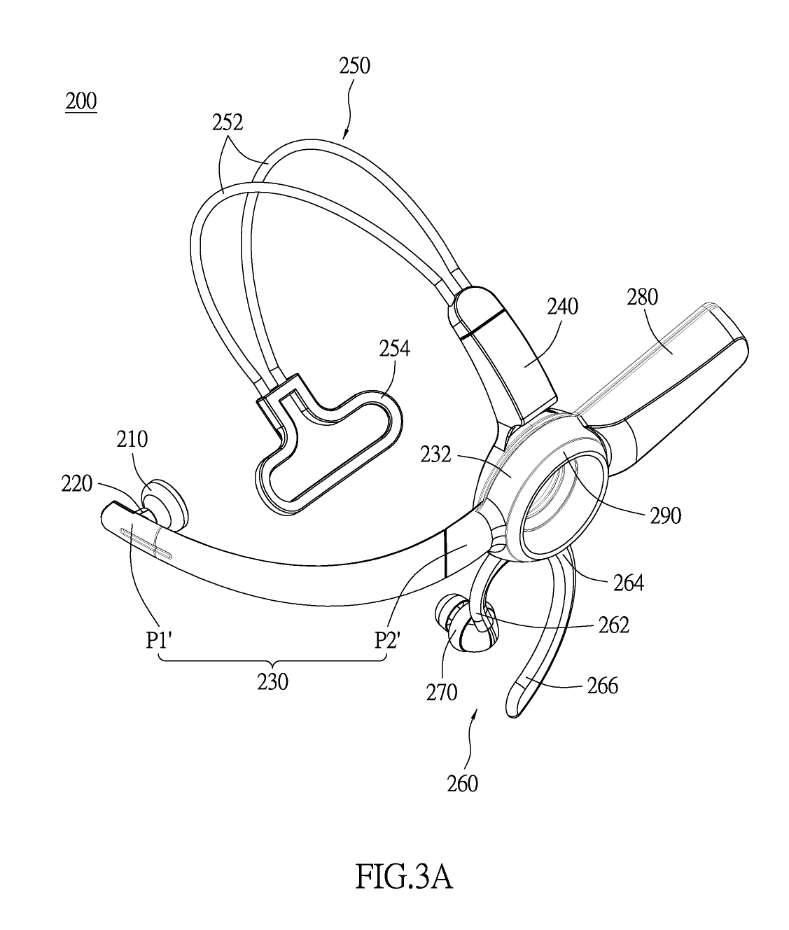

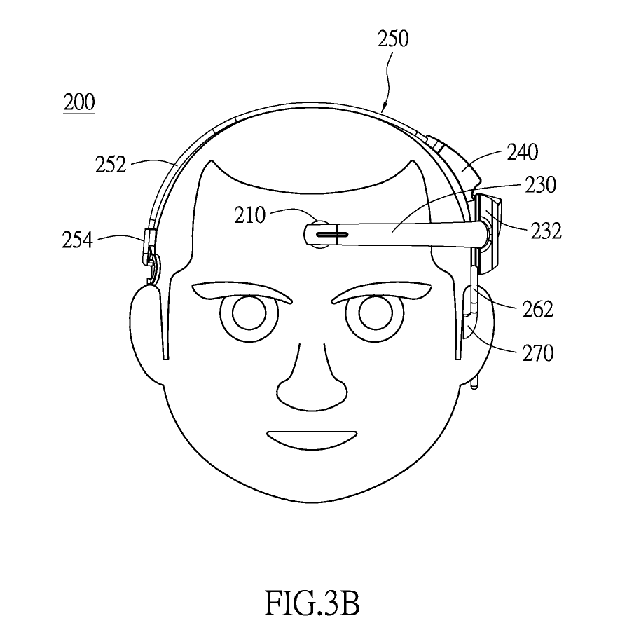

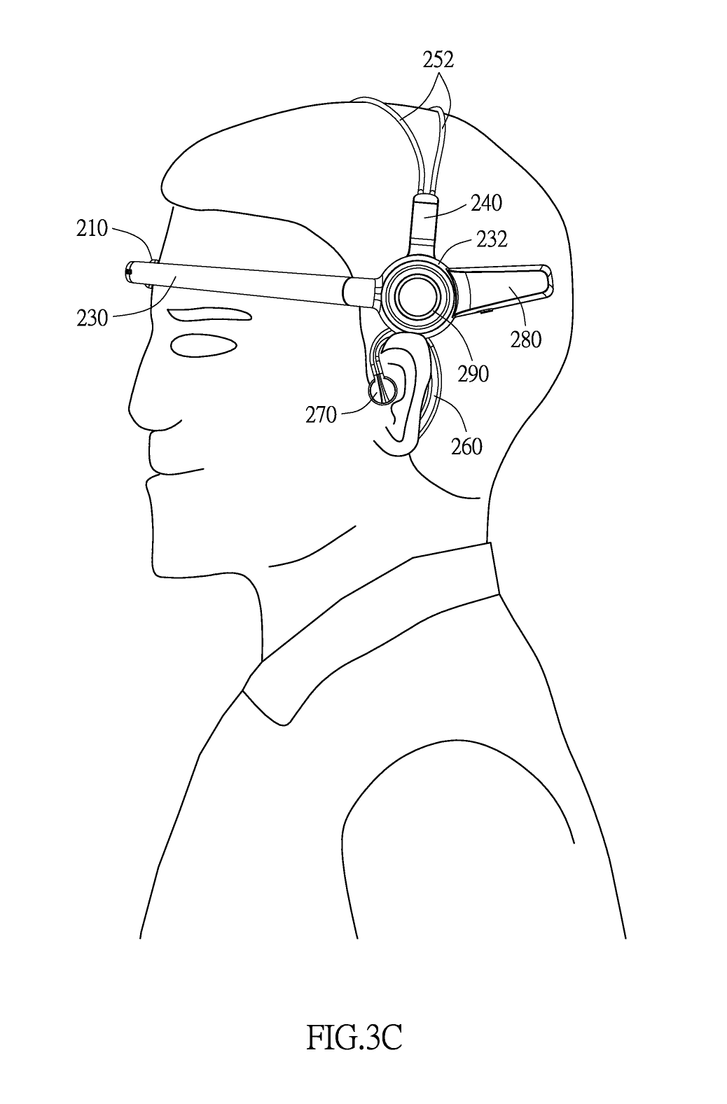

The sensing device for measuring electroencephalogram of the present disclosure includes a plurality of exemplary embodiments. The sensing devices for measuring electroencephalogram of disclosure exemplary embodiments are headband-type sensing devices for measuring electroencephalogram. A sensing device of one of exemplary embodiments may have only one sensing portion, and another sensing device of the other exemplary embodiment may have at least two sensing portions. The following detailed descriptions illustrate the above-mentioned sensing device for measuring electroencephalogram in accordance with FIG. 2A to 3C. Besides, the sensing device 100 and 200 for measuring electroencephalogram shown in FIG. 2A to 3C are merely provided for reference and illustration. The present disclosure is not limited to sensing devices 100 and 200.

FIG. 2A illustrates a perspective diagram of a sensing device for measuring electroencephalogram in accordance with the first exemplary embodiment of the present disclosure. Please refer to FIG. 2A. The sensing device 100 includes a sensing portion 110, a multi-directional joint 120, an arm 130, a connecting base 140 and a headband component 150. The sensing portion 110 is coupled to the multi-directional joint 120, and is disposed on the arm 130. The multi-directional joint 120 is coupled to the sensing portion 110 and the arm 130. The connecting base 140 is coupled to the arm 130 and the headband component 150, so that the arm 130 is coupled to the headband component 150 through the connecting base 140.

The arm 130 is a support where the sensing portion 110 is disposed. The arm 130 has a front end P1 and a back end P2 opposite to the front end P1. The sensing portion 110 is disposed on the front end P1 through the multi-directional joint 120. The back end P2 is coupled to the connecting base 140. Specifically, the arm 130 is arching and extends from the position above the ear to the forehead so that the sensing portion 110 could be in front of the forehead.

It is worth to mention that in the instant embodiment, the arm 130 could have flexibility so the arm 130 could be adjusted to cause that the sensing portion 110 is able to against the different head girth. Besides, the arm 130 could be made of plastic, metal, and so on. The present disclosure is not limited to the materials of the arm 130.

The sensing portion 110 is used to measure electroencephalogram from forehead, and then converts into electrical signals, namely generating a first electroencephalogram signal. The sensing portion 110 is coupled to the multi-directional joint 120. The multi-directional joint 120 could be a ball-and-socket joint. The multi-directional joint 120 is disposed on the arm 130, so that the sensing portion 110 can rotate with respect to the front end P1 of the arm 130 through the multi-directional joint 120. The sensing portion 110 could be against heads and foreheads with various shapes to attenuate the deviation of the measured electroencephalogram.

The headband component 150 includes a plurality of elastic arcuate bands 152 and an anti-slip pad 154. Each elastic arcuate band 152 has a first side E1 and a second side E2 opposite to the first side. The first side E1 is coupled to the anti-slip pad 154, and the second side E2 is coupled to the connecting base 140. Specifically, the elastic arcuate bands 152 are used to wear the top of a head. In the first exemplary embodiment, the headband component 150 includes two elastic arcuate bands 152. The distance between the adjacent elastic arcuate bands 152 is decrease from the middle of the elastic arcuate bands 152 to the first side E1 and the second side E2. In other exemplary embodiment, the quantity of the elastic arcuate bands 152 could be more than two. The present disclosure is not limited to the quantity of the elastic arcuate bands 152. Besides, the anti-slip pad 154 is bent alone the arch of the elastic arcuate bands 152 and coupled to the first side E1. The connecting base 140 is coupled to the second side E2. Therefore, the sensing device 100 is formed basically.

To be more precise, the elastic arcuate bands 152 have elasticity and toughness. The elastic arcuate bands 152 extend from one ear toward the other ear and pass through the top of head. The distance between the elastic arcuate bands 152 can let the headband component 150 fit the top of head. Therefore, the elastic arcuate bands 152 could match up to the head type of user, so that the headband component 150 could be worn steady on the top of the head. Besides, the elastic arcuate bands 152 could be made of elastic steel wires, elastic plastic, or elastic steel wires covered by rubber tubes. The present disclosure is not limited to the materials of the elastic arcuate bands 152.

The anti-slip pad 154 may be T-shaped (shown in FIG. 2A). The T-shaped anti-slip pad 154 has a connecting portion L1 and an anti-slip portion L2. The connecting portion L1 has two opposite sides, and the anti-slip portion L2 protrude from the two opposite sides. The connecting portion L1 connects the first side E1. The T-shaped anti-slip pad 154 is a soft pad, such as a rubber pad. The T-shaped anti-slip pad 154 is used to provide the headband component 150 for frictional force and improve comfort. While the T-shaped anti-slip pad 154 touches the hair or the scalp of the user closely, the headband component 150 is not easy to be slack. Besides, to prevent the headband component 150 from slack, the T-shaped anti-slip pad 154 could have veined pattern to enhance the frictional force. The present disclosure is not limited to the materials and structure of the T-shaped anti-slip pad 154.

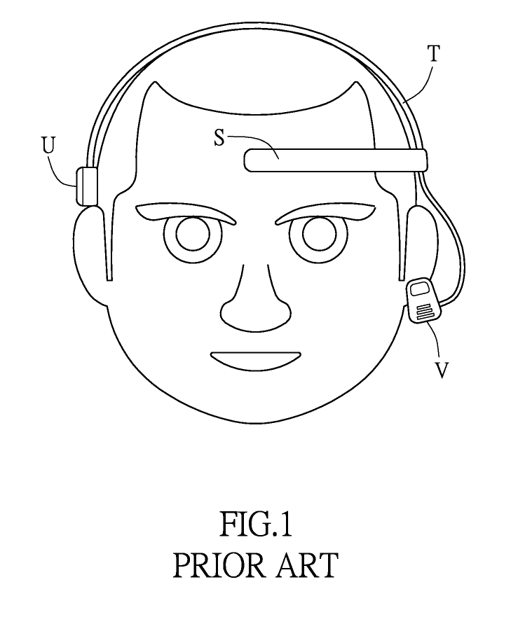

According to the above-mentioned exemplary embodiment, compared with conventional the plastic solid headband of electroencephalogram sensing device (for example, the headband T shown in FIG. 1), the elastic arcuate bands 152 could fit type of head more. Therefore, the sensing portion 110 could be hard to sliding down, and the elastic arcuate bands 152 are more steady and do not make the used fill tensed), so that the user could wear the headband component 150 comfortably. Compared with the foam anti-slip pad U, the design of T-shaped anti-slip pad 154 could provide more comfort and frictional force, so that the headband component 150 is worn steady.

FIG. 2B depicts a front view diagram of a sensing device for measuring electroencephalogram shown in FIG. 2A. FIG. 2C depicts a vertical view diagram of using a sensing device for measuring electroencephalogram shown in FIG. 2A. Please refer to FIG. 2B.and FIG. 2C, Explicitly, when using electroencephalogram sensing device 100, first, the user wears the elastic arcuate bands 152 so that it is on top, and then adjusts the anti-slip pad 154 to make sure it is positioned on an ear position so that it could not slip. Next, the arm 130 is adjusted to be in front of the forehead. The sensing portion 110 could be rotated relative to the front end P1 of the arm 130 through the multi-directional joint 120 so that the sensing portion 110 is against forehead. Therefore, the sensing portion 110 can measure the electroencephalogram, so that the sensing portion 110 gets a first electroencephalogram signal.

It is worth to mention that, according to first electroencephalogram signal, the brain activity could be observed. The user could know the physiological information after analyzing the first electroencephalogram signal through an analyzing system (for example a computer, not shown). The user could use the physiological information in many respects, such as physical assessment, therapy or rehabilitation.

FIG. 3A illustrates a perspective diagram of a sensing device for measuring electroencephalogram in accordance with the second exemplary embodiment of the present disclosure. Please refer to FIG. 3A. The structure of a device 200 in accordance with second exemplary embodiment is similar to the sensing device 100 in accordance with first exemplary embodiment. For example, the sensing device 100 and 200 include sensing portions and headband components. However, there are some differences between the sensing device 100 and 200 for measuring electroencephalogram. The following detailed description explains the difference between the sensing devices 100 and 200, and the same features are basically not described again.

The sensing device 200 includes a sensing portion 210, a multi-directional joint 220, an arm 230, a connecting base 240 and a headband component 250. The headband component 250 includes a plurality of elastic arcuate bands 252 and an anti-slip pad. For example, the anti-slip pad 254 could be a T-shaped anti-slip pad 254. The sensing portion 210 is coupled to the multi-directional joint 220. The sensing portion 210 is disposed on the front end P1′ through the multi-directional joint 220. The arm 230 is coupled to the headband component 250 through the connecting base 240. The elastic arcuate bands 252, the sensing portion 210, and the multi-directional joint 220 could be the same as the elastic arcuate bands 152, the sensing portion 110, and the multi-directional joint 120 of the first exemplary embodiment of the present disclosure. The difference between the headband component 250 and the headband component 150 is that T-shaped anti-slip pad 254 is hollowed. Therefore, the T-shaped anti-slip pad 254 could provide more frictional force, so that the headband component 250 is not easy to be slack.

The arm 230 further includes a pivot portion 232. The pivot portion 232 is disposed on the back end P2′ and coupled to the connecting base 240. Therefore, the arm 230 can rotate with respect to the headband component 250 through the pivot portion 232. Furthermore, the pivot portion 232 could be a pivot between the arm 230 and the headband component 250. Hence, the arm 230 can rotate with respect to the elastic arcuate bands 252 through the pivot portion 232, so that the angle between the arm 230 relative to the headband component 250 could be adjusted.

The sensing device 200 includes an ear hanging type frame 260 and the sensing reference portion 270. The ear hanging type frame 260 is used to hook around an ear. The ear hanging type frame 260 has an ear-bud terminal 262, a free terminal 266, and a linked portion 264 between the ear-bud terminal 262 and the free terminal 266. The ear-bud terminal 262 is coupled to the sensing reference portion 270, and the linked portion 264 is coupled to the pivot portion 232. Therefore, the sensing reference portion 270 is used to measure electroencephalogram, so that the sensing reference portion 270 generates a second electroencephalogram signal.

FIG. 3B depicts a front view diagram of a sensing device for measuring electroencephalogram shown in FIG. 3A. FIG. 3C depicts a vertical view diagram of using a sensing device for measuring electroencephalogram shown in FIG. 3A. Please refer to FIG. 3B and FIG. 3C. Explicitly, the ear hanging type frame 260 could have elasticity and toughness. For example, the ear hanging type frame 260 could be formed by steel wires covered by rubber or plastic materials. The present disclosure is not limited to the sensing device for the material of the ear hanging type frame 260.

Therefore, the user hooks the ear hanging type frame 260 around ear so the ear hanging type frame 260 could not press the ear. The materials of the sensing reference portion 270 could include silicone gel, foam, and electric-conductive adhesive. The electric-conductive adhesive is covered by silicone gel and foam, or the materials of the sensing reference portion 270 could be formed by electric-conductive rubber. The present disclosure is not limited to the materials and structure of the sensing reference portion 270. Moreover, the sensing reference portion 270 is coupled to the ear-bud terminal 262, and the sensing reference portion 270 is able to be put into external auditory canal to measure the second electroencephalogram signal. The second electroencephalogram signal is used as a reference signal.

According to the above-mentioned exemplary embodiment, compared with the conventional the ear clip V of electroencephalogram sensing device (the ear clip V shown in FIG. 1), the ear hanging type frame 260 could provide the user comfort and steady.

The sensing device 200 could further include a processing module 280. The processing module 280 is coupled to the sensing portion 210 and sensing reference portion 270 respectively, so that the processing module 280 could perform calculation of analysis according to the first electroencephalogram signal S1 and the second electroencephalogram signal S2, for example, to generate a calculating value. The calculating value could be a difference value between first electroencephalogram signal S1 and the second electroencephalogram signal S2. Specifically, the sensing portion 210 and the sensing reference portion 270 measure the electroencephalogram signals from the forehead and the ear respectively, and convert them to electrical signals, namely the first electroencephalogram signal S1 and the second electroencephalogram signal S2. The processing module 280 receives the first electroencephalogram signal S1 and the second electroencephalogram signal S2, so that the processing module 280 could calculate and analyze the difference value according to the first electroencephalogram signal S1 and the second electroencephalogram signal S2. Therefore, the user could know the physiological information used in many respects, such as physical assessment, therapy or rehabilitation.

Besides, in respect of practical application, the processing module 280 may include a printed circuit board (not shown) and at least one chip (not shown). The printed circuit board is coupled to the chip, and the chip could process the first electroencephalogram signal S1 and the second electroencephalogram signal S2. Moreover, to make the sensing device 200 have power to measure and to process the first electroencephalogram signal S1 and the second electroencephalogram signal S2, a battery device or a plug and receptacle design could be disposed in the processing module 280. The present disclosure is not limited to the processing module 280.

In other exemplary embodiment of the present disclosure, the processing module 280 could have hot plugging function. Hence, the processing module 280 could have an interface port, such as a universal serial bus (USB) port, a serial port, a console port, and the connecting base 240 could have a corresponding socket. Therefore, the processing module 280 could be inserted to the connecting base 240 to electrically connect to the sensing portion 210 and the sensing reference portion 270 through the interface port. Thus, the sensing portion 210 and the sensing reference portion 270 can transfer the first electroencephalogram signal S1 and the second electroencephalogram signal S2 to the processing module 280 through the interface port.

The sensing device 200 further includes a display module 290. The display module 290 is disposed on the pivot portion 232, and electrically coupled to the processing module 280. The display module 290 receives the calculating value from the processing module 280, such as the difference value between the first electroencephalogram signal S1 and the second electroencephalogram signal S2, so that the display module 290 displays the operate state of the sensing device 200. Therefore, the display module 290 could alert user by flashing light. The display module 290 could be a ring-shaped light-guide column, and the light source is a Light-Emitting Diode (LED), an Organic Light-Emitting Diode (OLED), or an incandescent lamp. Besides, the display module 290 could be a buzzer and shows the operate state of the sensing device 200 through sound. Therefore, the display module 290 is not shows the operate state through light. The present disclosure is not limited that the implementation method of the display module 290.

In summary, in the present disclosure, the sensing device for measuring electroencephalogram includes elastic arcuate bands. The elastic arcuate bands could match up to the head type of user, so that the elastic arcuate bands could be worn steady on the top of head. The T-shaped anti-slip pad could provide more comfort and frictional force, so that user could wear the headband component comfortably.

Besides, the sensing device further includes an ear hanging type frame disposed on the sensing reference portion. Therefore, compared with conventional the ear clip, the present disclosure provides users with frictional force and comfort.

The above-mentioned descriptions represent merely the exemplary embodiment of the present disclosure, without any intention to limit the scope of the present disclosure thereto. Various equivalent changes, alternations or modifications based on the claims of present disclosure are all consequently viewed as being embraced by the scope of the present disclosure.

What is claimed is:

1. A sensing device for measuring electroencephalogram comprising:
a connecting base;
an arm having a front end and a back end opposite to the front end, wherein the back end of the arm is coupled to the connecting base;
a multi-directional joint disposed on the front end of the arm;
a sensing portion coupled to the multi-directional joint, whereby the sensing portion can rotate with respect to the front end of the arm; and
a headband component comprising a plurality of elastic arcuate bands and an anti-slip pad, wherein each of the elastic arcuate bands has a first side and a second side opposite to the first side; the first side is coupled to the anti-slip pad, and the second side is coupled to the connecting base.

2. The sensing device for measuring electroencephalogram according to claim 1, wherein the anti-slip pad is a T-shaped anti-slip pad, the T-shaped anti-slip pad has an anti-slip portion and a connecting portion, the connecting portion has two opposite sides, the anti-slip portion protrudes from the two opposite sides, and the connecting portion connects the first side.

3. The sensing device for measuring electroencephalogram according to claim 2, wherein the T-shaped anti-slip pad is hollowed.

4. The sensing device for measuring electroencephalogram according to claim 1, wherein the adjacent elastic arcuate bands are separated by a distance, and the distance is decrease from the middle of the elastic arcuate bands to the first side and the second side.

5. The sensing device for measuring electroencephalogram according to claim 1, wherein the arm has a pivot portion, the pivot portion is disposed on the back end and connected to the connecting base, so that the arm can rotate with respect to the elastic arcuate bands through the pivot portion.

6. The sensing device for measuring electroencephalogram according to claim 5 further comprising an ear hanging type frame and a sensing reference portion, wherein the ear hanging type frame has an ear-bud terminal, a free terminal, and a linked portion between an ear-bud terminal and a free terminal, wherein the ear-bud terminal is coupled to the sensing reference portion, the linked portion is coupled to the connecting base, and the sensing reference portion is used to measure electroencephalogram.

7. The sensing device for measuring electroencephalogram according to claim 5, wherein the pivot portion has an open.

8. The sensing device for measuring electroencephalogram according to claim 6 further comprising a processing module, wherein the sensing portion is used to measure electroencephalogram from a forehead, so that the sensing portion generates a first electroencephalogram signal; the sensing reference portion is used to measure electroencephalogram from an ear, so that the sensing reference portion generates a second electroencephalogram signal, and the processing module is coupled to the sensing portion and sensing reference portion respectively, so a calculating value is generated by the processing module according to the first electroencephalogram signal and the second electroencephalogram signal.

9. The sensing device for measuring electroencephalogram according to claim 8 further comprising a display module, wherein the display module is couple to the processing module, the display module receive the calculating value, so that displaying the operating state of the sensing device for measuring electroencephalogram.
